(19) United States
(12) Patent Application Publication
(10) Pub. No.: US 2025/0090118 A1
(43) Pub. Date: Mar. 20, 2025

(54) Methods for Atherosclerotic Plaque Tissue Characterization in Spectral Computed Tomography

(71) Applicants: Wei Zhao, Jersey City, NJ (US); Changguo Ji, Cleveland, OH (US)

(72) Inventors: Wei Zhao, Jersey City, NJ (US); Changguo Ji, Cleveland, OH (US)

(21) Appl. No.: 18/500,075

(22) Filed: Nov. 1, 2023

(51) Int. Cl. A61B 6/50; A61B 6/00; A61B 6/03; A61B 6/46; G06T 11/00

(52) U.S. Cl. CPC A61B 6/504; A61B 6/03; A61B 6/468; A61B 6/482; A61B 6/5217; A61B 6/5258; G06T 11/008; G06T 2210/41

(57) ABSTRACT

Disclosed herein are methods for characterizing atherosclerotic plaque tissues in spectral computed tomography (CT). This invention employs dedicated spectral image reconstruction and vessel landmark-based fully motion correction algorithms to ensure high-quality spectral CT images. By quantifying the spectral attenuation characteristics of carotid plaque tissues and employing these attenuation values as discriminative filters across multiple spectral CT energy levels, the present invention enables a precise and consistent annotation process across various arteries at different body sites. This method handles a broad spectrum of plaque tissues and contrast variations observed across different spectral energy levels. This invention provides a robust and data-driven framework for characterizing atherosclerotic plaque tissues within the human arterial system, leveraging the unique tissue differentiation capabilities of spectral CT to enhance the accuracy and comprehensiveness of plaque tissue characterization.

### FIELD OF THE INVENTION

The present invention relates to methods for characterizing atherosclerotic plaque tissue in spectral computed tomography (CT) that produces imaging data at multiple energy levels and enables multi-energy plaque tissue characterization. Specifically, this invention includes quantitative medical imaging and analytics for atherosclerotic plaque tissues that encompass techniques for spectral CT image reconstruction, vessel motion correction, statistical analysis, and image annotation and segmentation.

### BACKGROUND OF THE INVENTION

Atherosclerosis is a complex and progressive condition marked by the accumulation of fatty deposits, cholesterol, inflammatory cells, and fibrous tissue within arterial walls. This chronic pathological condition poses a substantial global health concern and significantly contributes to cardiovascular disease, which is the foremost cause of death and disability globally. Precise evaluation and characterization of atherosclerotic plaque tissues within arteries are crucial for effective clinical management and therapeutic interventions.

The current diagnosis of atherosclerosis largely depends on conventional computed tomography (CT) for visualizing and evaluating plaques. However, conventional energy-integrating CT techniques face inherent challenges in precisely differentiating between plaque tissues. Given their reliance on a single, broad X-ray spectrum, conventional CT has limited ability to characterize atherosclerotic plaques. Importantly, there is substantial overlap in the X-ray attenuation profiles of primary plaque tissues including lipid-rich necrotic core (LRNC), intraplaque hemorrhage (IPH), and extracellular matrix (ECM). This makes it challenging to reliably differentiate between these tissue types. Therefore, conventional CT techniques impose critical limitations on quantitative characterization of plaque tissues.

Spectral CT techniques are new advances in CT imaging. They produce images at various spectral energy levels, allowing for a more detailed and enhanced characterization of plaque tissues based on their distinct energy absorption properties. Spectral CT can distinguish between different types of atherosclerotic plaque tissues in the arterial system by leveraging their unique energy absorption characteristics, which reflect their molecular compositions. Due to its sensitivity to energy absorption, spectral CT shows significant promise and potential for the quantitative characterization of atherosclerotic plaque tissues.

While plaque tissue annotation has been performed using histological examination of plaque tissues and tissue characterization models in prior arts (U.S. Pat. Nos. 11,087,460B2, 11,094,058B2, U.S. Pat. No. 11,120,312B2, and U.S. Pat. No. 11,715,187B2), this method suffers from inherent limitations and significant flaws. The use of co-registered plaque tissue samples for histological examination can lead to potential errors and cross-modality discrepancies when translating microscopic findings to clinical CT images. To address these issues, it is crucial to perform plaque annotation within the same imaging modality. Spectral CT is one such modality that can be effectively used for this purpose.

### SUMMARY OF THE INVENTION

The present invention introduces a new method for characterizing atherosclerotic plaque tissues using spectral CT imaging. The said methodology involves the quantification of attenuation characteristics inherent to plaque tissues, utilizing these quantified values (expressed in Hounsfield Unit; HU) as discriminative filters. This innovative approach enables an advanced and precise characterization process across various arteries at different body sites, capable of handling diverse plaque tissue types and contrast variations observed across multiple spectral energy levels. The disclosed method provides a robust, data-driven methodology for the comprehensive characterization of atherosclerotic plaque tissues. This invention embodies significant advancements in the field of cardiovascular disease diagnosis. Key features and innovations include:

1. Spectral CT application: This invention employs the unique capabilities of spectral CT, which captures multiple spectral energy levels of X-rays, enabling precise differentiation of plaque tissues based on their distinct energy absorption characteristics.

2. Image reconstruction and motion correction: This invention implements grayscale-constrained spectral iterative reconstruction coupled with vessel landmark-based fully motion correction to enhance image quality and reduce noise.

3. Baseline annotation: Images of carotid plaques serve as the benchmark for determining critical attenuation values. Carotid plaques are more readily identifiable due to factors such as their accessible neck location, larger size obstructing the artery, reduced patient motion artifacts, proximity to the body's surface, and dedicated CT protocols designed to enhance plaque visibility.

4. Determination of critical attenuation values: In some embodiments, the present invention integrates specialized analyses, plaque tissue differentiation, and statistical analysis to determine accurate critical attenuation values for various plaque tissues, including but not limited to lipid-rich necrotic cores (LRNC), intraplaque hemorrhage (IPH), calcification (CAL), and extracellular matrix (ECM).

5. Plaque characterization across various arteries at different body sites: The determined critical attenuation values act as discriminative filters for plaque tissue characterization across various arteries. In some embodiments, this process can involve the utilization of automatic segmentation techniques, including watershed and level set algorithms.

6. Weighted fusion for multi-energy segmentation: In some embodiments, plaque tissue characterization is performed in one or a subset of spectral energy levels with optimal contrast. This invention employs a weighted fusion approach to consolidate multi-energy segmentations, enhancing accuracy while minimizing noise.

The present invention offers a robust data-driven methodology for characterizing atherosclerotic plaque tissues. This methodology has substantial potential to significantly advance the field of cardiovascular disease diagnosis, thereby enhancing diagnostic accuracy and patient care.

### BRIEF DESCRIPTION OF THE FIGURES

FIG. 1 illustrates the overall workflow of plaque tissue characterization in spectral CT.

FIG. 2 illustrates the process of image preprocessing for baseline carotid plaque tissue annotation.

FIG. 3 illustrates the image selection process for baseline carotid plaque tissue annotation.

FIG. 4 illustrates the process of multi-energy carotid plaque tissue annotation.

FIG. 5 illustrates the process of determining critical attenuation values from multi-energy carotid plaque tissue annotations.

FIG. 6 illustrates the process of characterization for plaque tissues across various arteries at different body sites.

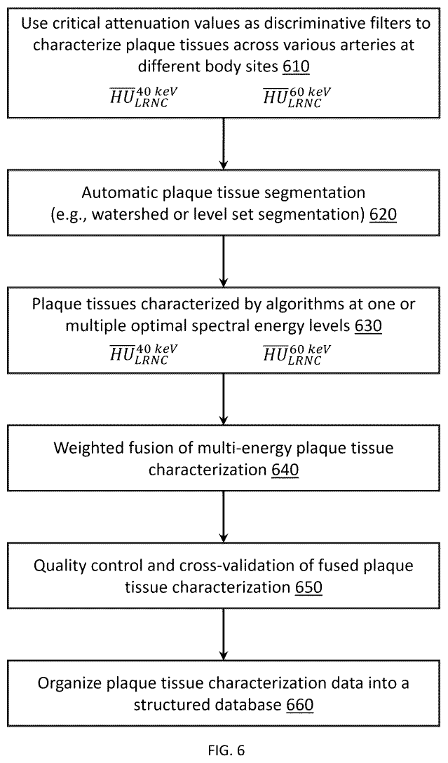

### DETAILED DESCRIPTION OF THE INVENTION

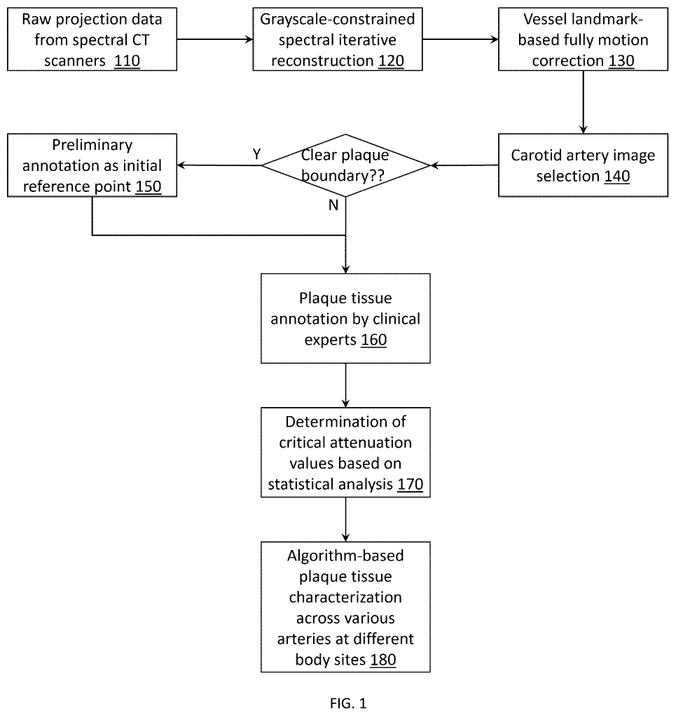

The present invention involves employing spectral CT scans, vessel landmark-based fully motion correction, statistical analysis, and automatic segmentation algorithms for the characterization of atherosclerotic plaque tissues within arteries. These spectral CT scans are derived from a variety of multi-energy CT systems capable of capturing the intricate interactions between X-rays and diverse atherosclerotic plaque tissues. Notably, this method encompasses exploiting photon-counting CT (PCCT) technology, which quantifies and assesses individual X-ray photons at various energy levels, enabling the precise differentiation of distinct plaque tissue types based on their unique energy absorption characteristics. FIG. 1 demonstrates the overall workflow of plaque tissue characterization in spectral CT. Details for each step in the workflow are explained below.

a. Raw Image Acquisition

The raw projection data from spectral CT scanners 110 are processed using advanced image reconstruction algorithms to generate spectral CT images at multiple spectral energy levels 120. In some embodiments, these algorithms employ mathematical modeling to estimate contrast agent concentration or other relevant parameters to differentiate between different plaque tissue types at each energy level. In some embodiments, grayscale-constrained spectral iterative reconstruction techniques are used to improve image quality and reduce noise. The reconstructed images form the basis for subsequent plaque tissue characterization and determination of critical attenuation values.

In some embodiments, spectral CT images can be collected from state-of-the-art CT scanner manufactures and all other comprehensive repositories of medical images, specifically focused on cardiovascular artery imaging. This repository can include data obtained from clinical studies, research databases, various medical institutions, etc.

b. Raw Image Preprocessing

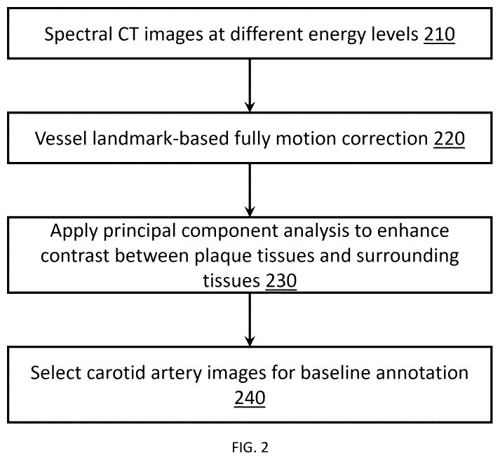

In some embodiments, the reconstructed spectral CT images necessitate initial preprocessing to optimize image quality while mitigating various motion artifacts, as illustrated in FIG. 2 This processing step involves the implementation of advanced vessel landmark-based fully motion correction algorithms 130, 220.

In some embodiments, to further enhance the quality of spectral CT images, principal component analysis is applied to enhance the contrast between atherosclerotic plaque tissues and their surrounding tissues 230.

In the process of baseline annotation, carotid plaque images are selected as the reference for this purpose 140, 240. Carotid plaques are more readily identifiable due to factors such as their accessible neck location, larger size obstructing the artery, reduced patient motion artifacts, proximity to the body's surface, and dedicated CT protocols designed to enhance plaque visibility. Regarding the biological compositions of atherosclerotic plaques, carotid plaques share similarities with plaques in other arterial locations. They all stem from atherosclerosis characterized by the accumulation of fatty deposits, cholesterol, inflammatory cells, and fibrous tissue within arterial walls. Therefore, plaque tissues in various arterial locations at different body sites not only share similar ranges of attenuation values but also have similar energy absorption characteristics on spectral CT scans.

c. Image Selection for Plaque Tissue Baseline Annotation

In an embodiment, the preprocessed carotid plaque images after vessel landmark-based fully motion correction 130, 310 undergo a rigorous selection process to ensure their accuracy in representing the spectrum of plaque types found in cardiovascular disease.

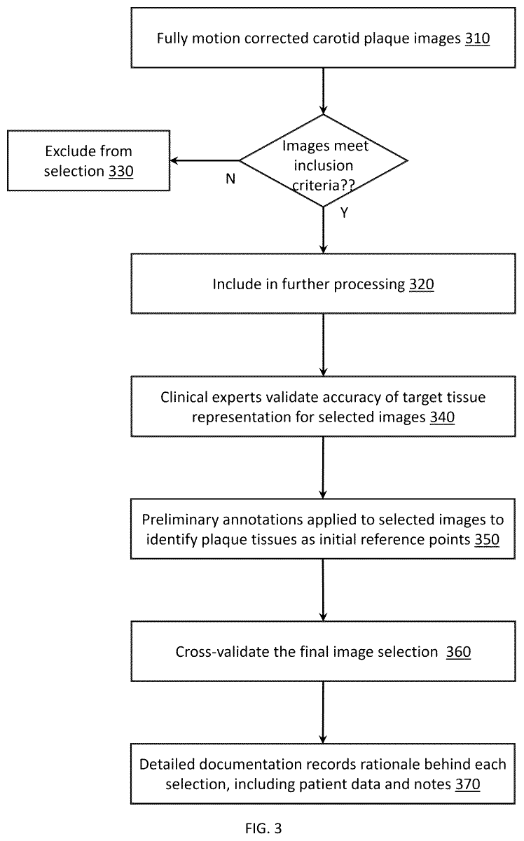

To ensure the highest image quality and clinical relevance, a rigorous selection process shown in FIG. 3 is implemented, guided by well-defined inclusion and exclusion criteria. Inclusion criteria can encompass prerequisites such as high-resolution imaging, availability of spectral energy levels that optimize plaque tissue contrast, clear and distinct tissue boundaries, and a diverse representation of atherosclerotic plaque tissues. Images that meet these stringent criteria are deemed suitable for subsequent processing 320.

In order to capture the full spectrum of atherosclerotic plaque tissue types, it is necessary to include images from a wide range of patient demographics, disease stages, and plaque types. The primary plaque types of interest typically encompass but not limited to lipid-rich necrotic cores (LRNC), intraplaque hemorrhage (IPH), calcification (CAL), and extracellular matrices (ECM).

In some embodiments, clinical experts in cardiovascular disease are involved to validate the selected images 340 and help confirm that the selected images accurately represent the targeted plaque tissues.

In some embodiments, preliminary annotation can be applied to selected images to identify boundaries of various plaque tissues 150, 350. These initial annotations serve as a reference point for the subsequent more detailed plaque tissue annotation process.

The final selection undergoes cross-validation 360 to ensure that the selected images are appropriate for subsequent baseline annotation. Importantly, this validation step occurs within the same image modality to maintain consistency and accuracy throughout the process.

The rationale for selecting each image is documented 370. This documentation can involve relevant patient data, including but not limited to age, gender, clinical history, physician notes, and any other pertinent data that contributes to plaque tissue annotation process.

d. Multi-Energy Carotid Plaque Tissue Annotation

Prior studies (Buckler et al., 2021; Karlöf et al., 2021; Buckler, 2022; Lal et al., 2022; Obuchowski and Buckler, 2022; Buckler, Doros, et al., 2023; Buckler, Gotto, et al., 2023; Buckler, Sakamoto, et al., 2023) have utilized an annotation method based on the examination using co-registered surgical histological sample complemented by trained tissue characterization models to characterize plaque tissues. However, this approach is not inherently derived from imaging data and often raises human-related errors, registration errors, and cross-modality discrepancies. Errors may arise during the translation of microscopic insights from histological examinations to clinical CT images. Histological processing and sectioning can introduce potential artifacts and tissue deformations that may deviate from the in vivo counterpart, resulting in structural variations and alignment disparities. Moreover, mapping from two-dimensional histology images to three-dimensional CT volumes can cause errors due to dimensional inconsistencies. Consequently, plaque annotation necessitates the use of the same imaging modality.

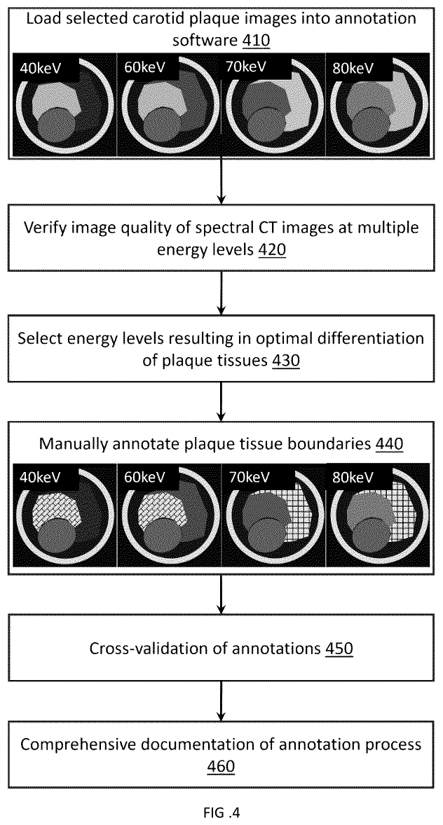

In the present invention, we present an annotation method based on spectral CT images 160 as illustrated in FIG. 4.

In some embodiments, the selected carotid plaque images are loaded into image annotation software 410, specifically designed for atherosclerotic plaque tissue characterization. The software provides users with essential tools, including zooming, panning, adjustment of imaging contrast and brightness, and manual drawing and editing of masks.

Clinical experts verify image quality of selected carotid spectral CT images 420 and select spectral energy levels that result in optimal differentiation between various plaque tissues 430. They focus on plaque tissues including but not limited to LRNC, IPH, CAL, and MAT. Grayscale images are visually assessed, and reference to density maps or other available data can be used to aid in making necessary adjustments.

For a specific plaque tissue, clinical experts employ the manual annotation tool within the software to delineate the boundaries of the entire plaque tissue on the images 440. This manual annotation can occur in one energy level or multiple energy levels that optimize tissue differentiation.

The annotations undergo a cross-validation process 450. The process can involve comparing annotations across multiple spectral energy levels and verifying delineated plaque boundaries to ensure precise placement and coverage of the targeted plaque tissues.

The annotation process can involve comprehensive documentation encompassing plaque tissue identification, a detailed rationale for each annotation, the selected energy levels, and any observations or challenges encountered 460.

By integrating the expertise of clinical experts, the unique multi-energy capabilities of spectral CT, and precise multi-energy annotation, this plaque tissue annotation process establishes a robust foundation to ensure the accuracy of subsequent plaque tissue characterization. It optimizes the potential of spectral CT to distinguish between atherosclerotic plaque tissues enabling robust characterization.

e. Determination of Critical Attenuation Values from Multi-Energy Carotid Plaque Tissue Annotation

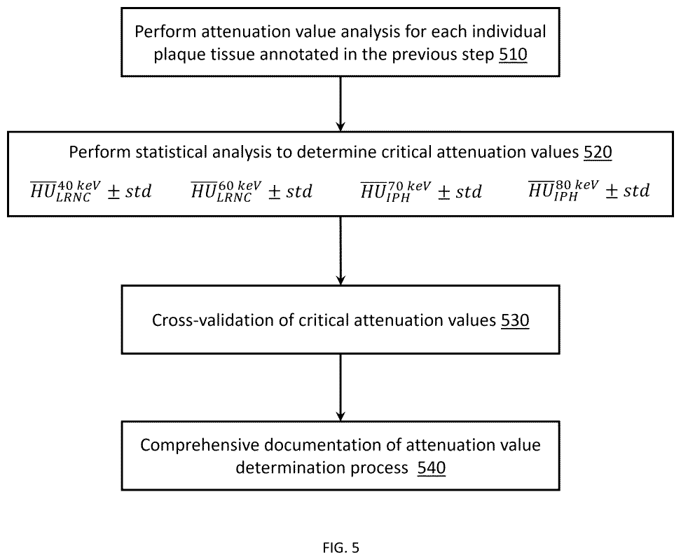

The process of determining critical attenuation values from multi-energy carotid plaque tissue annotations 170 is illustrated in FIG. 5. In some embodiments, determination of critical attenuation values is performed separately for each plaque tissue annotated in the previous step. In the scenario where multiple plaque tissues exist within a single carotid artery image, each tissue undergoes individual attenuation value analysis 510.

In some embodiments, statistical analysis can be performed to determine the critical attenuation values for the annotated plaque tissues 520. The analysis can involve analyzing the distribution of spectral data within each plaque tissue and performing hypothesis testing to compare means of attenuation values of different plaque tissues. Statistical parameters, including but not limited to, mean, standard deviation, percentile values, and others can be calculated to characterize the spectral properties of each type of plaque tissue.

The critical attenuation values determined through the previous step undergo a cross-validation process 530. This validation step involves comparing the attenuation values obtained from different energy levels and analytical methods.

The process of determining attenuation values can involve detailed documentation with a transparent record of the methods, procedures, and results 540. This documentation encompasses the rationale for the selection of optimal energy levels, specialized analyses of spectral CT images, tissue differentiation procedures, and statistical methods utilized.

The method for determining critical attenuation values through comprehensive analyses of spectral CT data, multi-energy tissue annotation, and statistical analysis take advantage of the unique multi-energy capability of spectral CT to precisely quantify the unique attenuation signature of each plaque tissue. The ability of spectral CT to differentiate plaque tissues based on energy-dependent attenuation provides a robust quantitative basis for plaque tissue characterization and risk assessment. This enhances plaque tissue characterization beyond the capabilities of conventional energy-integrating CT.

f. Plaque Tissue Characterization Across Various Arteries at Different Body Sites

In this crucial step 180, the critical attenuation values derived from the previous step using carotid plaque images act as discriminative filters for the characterization of plaque tissues across various arteries at different body sites 610. These critical attenuation values are utilized as reference points in one or multiple spectral energy levels that optimize tissue differentiation. This characterization process can be conducted through automatic segmentation methods 620.

In some embodiments, the watershed segmentation algorithm can be employed. During the process of identifying an LRNC in coronary artery, the critical attenuation values are initially used as discriminative filters to identify the preliminary plaque tissue boundary and generate seed points for the subsequent step. The initial filtering based on the critical attenuation values reject obvious background regions to focus the watershed algorithm on the most potential plaque locations. Subsequently, the watershed segmentation subdivides the initial boundary into homogeneous regions with more defined boundaries. The watershed algorithm then incorporates image intensity and gradient information to refine the plaque tissue boundaries. This process can encompass edge smoothing, region merging or splitting.

In some embodiments, an alternative automatic segmentation algorithm that can be employed is level set segmentation. During the process of identifying an LRNC in coronary artery, the critical attenuation values are initially employed as discriminative filters to generate preliminary boundaries that enclose image voxels within the potential plaque tissue. This captures broad plaque regions with possibly blurred and indistinct boundaries. Subsequently, a level set contour is initialized near the boundaries of the initially selected plaque region. The algorithm then evolves the level set curve outwards until reaching the plaque boundaries determined by the attenuation value filters. Finally, the level set deforms based on the underlying energy calculated by the energy function to fit the true plaque contours. The resulting segmentation encompasses the voxels enclosed within the converged level set boundaries.

The segmentation process can be performed within one or multiple spectral energy levels that optimize tissue differentiation 630. The segmentation approaches are not limited to the aforementioned techniques, and any other appropriate segmentation techniques can be employed in conjunction with the initialization of attenuation value filters.

In some embodiments, the plaque tissue characterization is performed in one or multiple spectral energy levels. To consolidate the multi-energy characterization into a final integrated optimal plaque tissue characterization, a weighted fusion approach is employed 640. Energy levels that optimize plaque tissue boundary differentiation are assigned higher weights, while noisy levels with relatively poor differentiation are allocated lower weights. Voxel-wise weighted averaging is applied across energy levels, biased towards high-differentiation energy levels, to generate the consolidated plaque characterization. Mathematical optimization algorithms fine-tune the weights of energy levels to maximize characterization accuracy.

Regardless of the image segmentation methods employed, rigorous quality control measures are applied to validate the accuracy of the final fused characterization 650. This ensures that the fused plaque tissue boundaries align with anatomical and clinical characteristics of the respective imaged plaque tissues. Necessary adjustments can be made to achieve precise plaque tissue characterization.

The final fused characterization data, which encompasses plaque types, anatomical locations, attenuation values, and patient demographics information and disease stages, are systematically organized into a structured database 660.

The present invention optimizes the characterization of atherosclerotic plaque tissues across various arteries, accounting for a broad range of plaque tissues and differentiation across different spectral energy levels. The disclosed techniques may be modified or adapted in various ways without deviating from the scope of the present invention. The specification and drawings are therefore to be regarded as illustrative rather than restrictive. The following claims aim to encompass all modifications, equivalents, and alternatives of the described embodiments that would be apparent to one of ordinary skills in the art. Any recited features, capacities elements, components, and applications are intended to be exemplary rather than limiting, and the invention is defined solely by the appended claims.

The term “comprising” as used in the claims does not exclude other methods, steps, or procedures. The term “a” or “an” as used in the claims does not exclude a plurality. A method may fulfill the function of several methods recited in the claims.

1. A method for preparing multi-energy spectral computed tomography (CT) images for atherosclerotic plaque analysis, the method comprising: acquisition of raw spectral CT projection data from patients who undergo cardiovascular disease diagnosis; reconstruction of spectral CT images from said raw projection data acquired from spectral CT scanners; application of motion correction algorithms to remove motion artifacts; and selection of carotid plaque spectral CT images for plaque tissue annotation.

2. The method of claim 1 wherein said raw spectral CT projection data comprises energy-resolving photon-counting data or image projections acquired at a plurality of spectral energy levels.

3. The method of claim 1 wherein the method for reconstructing spectral CT images comprises grayscale-constrained spectral iterative reconstruction techniques.

4. The method of claim 1 wherein said motion correction methods comprise vessel landmark-based fully motion correction algorithms.

5. The method of claim 1, further comprising the application principal component analysis as an image processing technique to facilitate plaque tissue differentiation.

6. The method of claim 1 wherein the selection of carotid plaque spectral CT images comprises the application of a plurality of inclusion criteria.

7. The method of claim 1 wherein the selection of carotid plaque spectral CT images comprises incorporation of preliminary atherosclerotic plaque tissue annotation.

8. A method for determining critical attenuation values from annotated carotid plaque tissues, the method comprising: manual annotation of carotid plaque tissue boundaries at optimal spectral energy levels; cross-validation of said manual plaque annotations; application of statistical analysis on spectral CT data to determine critical attenuation values for annotated carotid plaque tissues; and cross-validation of said critical attenuation values.

9. The method of claim 8 wherein manual annotation of carotid plaque tissues utilizes dedicated annotation software.

10. The method of claim 8 wherein the manual annotation comprises delineation of boundaries of plaque tissues including but not limited to lipid-rich necrotic core (LRNC), intraplaque hemorrhage (IPH), calcification (CAL), and extracellular matrix (ECM).

11. The method of claim 8 wherein manual annotation of carotid plaque tissues is performed by clinical experts in cardiovascular imaging.

12. The method of claim 8 wherein said critical attenuation values comprise the attenuation values that delineate a plaque tissue from its surrounding tissues.

13. The method of claim 8 wherein statistical analysis is conducted for each individual annotated carotid plaque tissue.

14. The method of claim 8 wherein statistical analysis is conducted at spectral energy levels that optimize plaque tissue differentiation.

15. The method of claim 8 wherein statistical analysis comprises but not limited to evaluation of distribution, mean, median, standard deviation, and percentile values.

16. A method for characterizing atherosclerotic plaque tissues across various arteries at different body sites, the method comprising: filtering spectral CT images based on said critical attenuation values; application of automatic segmentation of atherosclerotic plaque tissues guided by said filtering; cross-validation of the multi-energy plaque tissue characterization; and fusion of multi-energy plaque characterizations by weighted averaging algorithms.

17. The method of claim 16 wherein filtering uses said critical attenuation values derived from carotid plaque analysis as reference values.

18. The method of claim 16 wherein the automatic plaque tissue segmentation methods comprise but not limited to image segmentation methods based on watershed and level set algorithms.

19. The method of claim 16 wherein automatic plaque tissue segmentation is constrained within the boundaries generated by multi-energy filtering.

20. The method of claim 16 wherein weighted averaging fuses atherosclerotic plaque boundaries delineated across optimized spectral energy levels.
